(19) United States
(12) Patent Application Publication
(10) Pub. No.: US 2026/0240921 A1
(43) Pub. Date: Aug. 20, 2026

(54) NOVEL BACILLUS VELEZENSIS DIRECT-FED MICROBIAL ISOLATE

(71) Applicant: KEMIN INDUSTRIES, INC., Des Moines, IA (US)

(72) Inventors: Michael BELVERUD, Pleasant Hill, IA (US); Mitchell POSS, Johnston, IA (US); Hannah PIPER, Tomah, WI (US)

(73) Assignee: KEMIN INDUSTRIES, INC., Des Moines, IA (US)

(21) Appl. No.: 19/544,838

(22) Filed: Feb. 19, 2026

Related U.S. Application Data
(60) Provisional application No. 63/760,478, filed on Feb. 19, 2025.

(51) Int. Cl. A61K 35/742; A23K 10/18; A61K 9/00; A61K 35/747

(52) U.S. Cl. CPC A61K 35/742; A23K 10/18; A61K 9/0019; A61K 9/0053; A61K 35/747

(57) ABSTRACT

The present invention relates to a novel B. velezensis strain that improves the health and performance of animals. Another aspect of the present invention relates to using the novel strain to inhibit or reduce pathogen growth in animal feed, for instance as an animal feed additive. Another aspect of the present invention relates to a direct-fed microbial composition, wherein administration of the composition results in an improved performance parameter. Another aspect of the present invention relates to a novel strain that can be incorporated into food or foodstuff.

### CROSS-REFERENCE TO RELATED APPLICATIONS

This application claims priority to U.S. Provisional Application 63/760,478, NOVEL BACILLUS VELEZENSIS DIRECT-FED MICROBIAL ISOLATE.

### REFERENCE TO A DEPOSIT OF BIOLOGICAL MATERIAL

This application contains a reference to a deposit of biological material, which deposit is incorporated by reference.

### FIELD OF THE INVENTION

The present invention relates to a novel Bacillus velezensis strain and its use as a direct-fed microbial or probiotic.

### BACKGROUND OF THE INVENTION

Direct-fed microbials (DFMs), often called probiotics, are intended to improve the intestinal health of the animals that consume them. An ideal isolate should be able to withstand the high temperatures (80-90° C.) of pelleting, protect against pathogens, and improve performance metrics such as bodyweight gain and feed efficiency. Key criteria for any new isolate that may be commercialized are strong in vitro efficacy against numerous pathogens, and production of enzymes that can aid in digestion. The use of Bacillus strains as probiotic ingredients in the feed industry is well known in the art. The gram-positive spore-forming bacteria of the genus Bacillus have been favored because of their ability to survive pelleting at high temperatures (90° C.).

Enzymes produced and secreted by probiotics can be beneficial to livestock by hydrolyzing molecules that can be absorbed through the intestines. Amylase, xylanase, protease, and lipase break down starch, xylan, protein, and fats into glucose, xylose, amino acids, and free-fatty acids. These probiotic-produced enzymes can improve feed efficiency and growth performance.

There is therefore a need for DFMs which influence the gut microflora in a positive way and assist with digestion of specific feed ingredients.

For these and other reasons, there is a need for the present invention.

### SUMMARY OF THE INVENTION

The present invention relates to the discovery that the addition of a DFM from Bacillus velezensis species to animal feed can be used to prevent and/or control livestock pathogens, including E. coli, Salmonella, Streptococcus, Rhodocoous, Enterococcus, Clostridium, and other common livestock pathogens. The Bacillus velezensis species can also improve the body weight gain and/or feed efficiency.

In a first aspect the invention relates to a Bacillus velezensis strain characterized in that:
i) the Bacillus velezensis strain has high levels of amylase, protease, lipase, and xylanase activity;
ii) the Bacillus velezensis strain has enzymatic activity under aerobic and/or anaerobic conditions that hydrolyzes one or more substrates selected from the group consisting of starch, xylan, protein, and fats into glucose, xylose, amino acids, and free-fatty acids;
iii) the Bacillus velezensis strain shows strong in vitro inhibition of common livestock pathogens, including E. coli, Salmonella, Streptococcus, Rhodocaccus, Enterococcus, and Clostridium; and
iv) the Bacillus velezensis strain improves feed efficiency and/or growth performance in livestock and chickens fed with the Bacillus velezensis strain.

The invention further relates to the novel strain having the deposit accession number PTA-127793; a strain having all of the identifying characteristics of Bacillus velezensis N23 or a mutant thereof.

The present invention also relates to compositions comprising the Bacillus velezensis strain according to the invention which improves health and performance of production animals.

In a preferred embodiment the invention relates to a composition, e.g., comprising a carrier and a Bacillus velezensis strain wherein:
i) the Bacillus velezensis strain has high levels of amylase, protease, lipase, and xylanase activity;
ii) the Bacillus velezensis strain has enzymatic activity under aerobic and/or anaerobic conditions that hydrolyzes one or more substrates selected from the group consisting of starch, xylan, protein, and fats into glucose, xylose, amino acids, and free-fatty acids;
iii) the Bacillus velezensis strain shows strong in vitro inhibition of common livestock pathogens, including E. coli, Salmonella, Streptococcus, Rhodococcus, Enterococcus, and Clostridium;
iv) the Bacillus velezensis strain improves feed efficiency and/or growth performance in livestock and/or chickens fed with the Bacillus velezensis strain; and
v) the Bacillus velezensis strain having the deposit accession number PTA-127793; a strain having all of the identifying characteristics of Bacillus velezensis PTA-127793 or a mutant thereof.

### DETAILED DESCRIPTION OF THE INVENTION

Before the present invention is described in further detail, it is to be understood that the invention is not limited to the particular embodiments described. It is also to be understood that the terminology used herein is for the purpose of describing particular embodiments only, and is not intended to be limiting. The scope of the present invention will be limited only by the claims. As used herein, the singular forms “a”, “an”, and “the” include plural embodiments unless the context clearly dictates otherwise.

It should be apparent to those skilled in the art that many additional modifications beside those already described are possible without departing from the inventive concepts. In interpreting this disclosure, all terms should be interpreted in the broadest possible manner consistent with the context. Variations of the term “comprising”, “including”, or “having” should be interpreted as referring to elements, components, or steps in a non-exclusive manner, so the referenced elements, components, or steps may be combined with other elements, components, or steps that are not expressly referenced. Embodiments referenced as “comprising”, “including”, or “having” certain elements are also contemplated as “consisting essentially of” and “consisting of” those elements, unless the context clearly dictates otherwise. It should be appreciated that aspects of the disclosure that are described with respect to a system are applicable to the methods, and vice versa, unless the context explicitly dictates otherwise.

Numeric ranges disclosed herein are inclusive of their endpoints. For example, a numeric range of between 1 and 10 includes the values 1 and 10. When a series of numeric ranges are disclosed for a given value, the present disclosure expressly contemplates ranges including all combinations of the upper and lower bounds of those ranges. For example, a numeric range of between 1 and 10 or between 2 and 9 is intended to include the numeric ranges of between 1 and 9 and between 2 and 10.

The present invention is directed to bacteria that are effective at inhibiting the growth of E. coli and other pathogens in livestock.

N23 is a novel probiotic microorganism. The strain was deposited on Jun. 4, 2024, under the terms of the Budapest Treaty on the International Recognition of the Deposit of Microorganisms for the Purpose of Patent Procedure. Deposit was made by Kemin Industries, Inc. at the ATCC Patent Depository 10801 University Boulevard, Manassas, Virginia 20110 USA, which assigned it Accession Number PTA-127793.

In a first embodiment, the current invention is directed to a Bacillus velezensis strain and preparations that contain or are derived from this strain or mutants thereof. Specifically, the invention is directed to one or more of the following groups:
a) the Bacillus velezensis strain deposited under PTA-127793 at the ATCC;
b) a mutant of the Bacillus velezensis strain deposited under PTA-127793 having all identifying characteristics of the strain PTA-127793, wherein the mutant preferably has a DNA sequence identity to PTA-127793 of at least 95%, preferably at least 96, 97 or 98%, and more preferably at least 99 or 99.5%;
c) a preparation containing (a) or (b);
d) a preparation containing an effective mixture of metabolites from (a), (b) or (c).

The strain of the current invention and mutants thereof is preferably characterized by at least one, and more preferably by all, of the following further features:
i) the Bacillus velezensis strain has high levels of amylase, protease, lipase, and xylanase activity;
ii) the Bacillus velezensis strain has enzymatic activity under aerobic and/or anaerobic conditions that hydrolyzes one or more substrates selected from the group consisting of starch, xylan, protein, and fats into glucose, xylose, amino acids, and free-fatty acids;
iii) the Bacillus velezensis strain shows strong in vitro inhibition of common livestock pathogens, including E. coli, Salmonella, Streptococcus, Rhodococcus, Enterococcus, and Clostridium;
iv) the Bacillus velezensis strain reduces the need for antibiotics;
v) the Bacillus velezensis strain improves feed efficiency and/or growth performance in livestock and/or chickens fed with the Bacillus velezensis strain.

The strain is preferably further characterized by a lipase activity of at least 150 U/L, more preferably at least 151 U/L, amylase activity of at least 700 U/L, more preferably at least 716 U/L, protease activity of at least 40 μg/mL, more preferably at least 46 μg/mL, and xylanase activity of at least 100 U/mL, more preferably at least 108 U/mL.

In addition, the strains of the current invention preferably survive the high temperatures necessary for pelleting animal feed, in particular, they preferably survive a temperature of at least 80° C. for at least 20 minutes.

Without wishing to be bound by any theory, it is thought that the Bacillus velezensis strain according to the current invention enhance animal health by a multifaceted mode of action, including the production and secretion of enzymes that can then break down starch, xylan, protein, and fats into glucose, xylose, amino acids, and free-fatty acids, thereby improving feed efficiency and growth performance. The Bacillus velezensis strain further competes with pathogenic bacteria by consuming the available nutrients, thereby suppressing effective establishment of pathogenic bacteria in the gut.

It is an advantage of DFMs in comparison to antibiotics, that they do not destroy bacteria indiscriminately and do not they lead to antibiotic resistant strains of pathogenic bacteria. Normally, they are able to selectively compete with pathogenic bacteria by production of antimicrobial substances with specific efficacy, and are ideally able to simultaneously enhance the growth and viability of beneficial gut microflora. Further, they are preferably able to stimulate a systemic immune response in the treated animals.

The mutant strains of PTA-127793 of the current invention are preferably spontaneous mutants. The term “spontaneous mutant” refers to mutants that arise from PTA-127793 without the intentional use of mutagens. Such spontaneous mutants may be obtained by classical methods, such as growing the Bacillus velezensis strain in the presence of UV light or in the presence of a certain antibiotic to which the parent is susceptible and testing any resistant mutants for improved biological activity or improved ability to enhance one or more of the indicia of animal health. Other methods for identifying spontaneous mutants are known to those of ordinary skill in the art. But besides these preferred spontaneous mutants all other kinds of mutants of PTA-127793, e.g., mutants obtained by genetic engineering, are also part of the current invention.

One particular embodiment of the current invention are mutants of the strain PTA-127793 that are not found in nature and that have the characteristics described herein.

In a preferred embodiment of the current invention, the strains and preparations of the present invention are administered orally to animals, including livestock and production animals. In alternative embodiments, the strains and preparations of the present invention are administered orally to humans.

Thus, a further subject of the current invention are compositions, such as feedstuffs, foodstuffs, drinking and rearing water as well as therapeutic compositions, containing a B. velezensis strain and/or a preparation of the current invention.

Another aspect of the present invention relates to use of a B. velezensis strain and/or a preparation as a direct-fed microbe (DFM) or probiotic ingredient in feed or food products.

Preferred foodstuffs according to the invention include dairy products, in particular yogurt, cheese, milk, butter and quark.

The cells of the strains of the current invention may be present, in particular in compositions as spores (which are dormant), as vegetative cells (which are growing), as transition state cells (which are transitioning from growth phase to sporulation phase) or as a combination of at least two, and preferably all of these types of cells. In a preferred embodiment, the composition of the current invention comprises mainly or only spores. In some embodiments, the invention comprises a biologically pure culture of B. velezensis. In some embodiments, the invention comprises an isolated B. velezensis strain.

The Bacillus velezensis strain of the current invention and compositions containing them, when administered to animals, preferably enhance the health of such animals and/or improve the general physical condition of such animals and/or improve the feed conversion rate of such animals and/or decrease the mortality rate of such animals and/or increase the survival rates of such animals and/or improve the weight gain of such animals and/or increase the productivity of such animals and/or increase the disease resistance of such animals and/or increase the immune response of such animals and/or establish or maintain a healthy gut microflora in such animals and/or reduce antibiotic use in such animals and/or reduce the pathogen shedding through the feces of such animals. In particular, the strains and compositions of the current invention might be used to assist in re-establishing a healthy balance of the gut microflora after administration of antibiotics for therapeutic purposes.

A further subject of the current invention is therefore a method of enhancing the health of animals and/or of improving the general physical condition of animals and/or of improving the feed conversion rate of animals and/or of decreasing the mortality rate of animals and/or of increasing the survival rates of animals and/or of improving the weight gain of animals and/or of increasing the productivity of animals and/or of increasing the disease resistance of animals and/or of increasing the immune response of animals and/or of establishing or maintaining a healthy gut microflora in animals and/or reduce antibiotic use in animals and/or of reducing the pathogen shedding through the feces of animals, wherein the strains and/or preparations of the current invention or the compositions of the current invention, which comprise such strain(s), are administered to animals.

A further subject of the current invention is therefore also the use of strains and/or preparations and/or compositions of the current invention for enhancing the health of animals and/or for improving the general physical condition of animals and/or for improving the feed conversion rate of animals and/or for decreasing the mortality rate of animals and/or for increasing the survival rates of animals and/or for improving the weight gain of animals and/or for increasing the productivity of animals and/or for increasing the disease resistance of animals and/or for increasing the immune response of animals and/or for establishing or maintaining a healthy gut microflora in animals and/or reduce antibiotic use in animals and/or for reducing the pathogen shedding through the feces of animals, wherein the strains and/or preparations of the current invention or the compositions of the current invention, which comprise such strain(s), are administered to animals.

Another aspect of the present invention relates to administering a novel strain as described herein, including but not limited to use as a direct-fed microbial composition, containing the strain or mutants thereof, for enhancing the health of animals and/or for improving the general physical condition of animals and/or for improving the feed conversion rate of animals and/or for decreasing the mortality rate of animals and/or for increasing the survival rate of animals and/or for improving the weight gain of animals and/or for increasing the productivity of animals and/or for increasing the disease resistance of animals and/or for increasing the immune response of animals and/or for establishing or maintaining a healthy gut microflora in animals and/or reduce antibiotic use in animals and/or for reducing the pathogen shedding through the feces of animals.

“Increasing the productivity of animals” may refer, in particular, to any of the following: production of more or higher quality eggs, milk or meat or increased production of weaned offspring. Such increase maybe measured by any standard metric or performance parameter, including but not limited to weight gain, European production efficacy factor, feed conversion ratio, or egg yield.

The methods and uses of the strains, preparations and compositions of the current invention can be therapeutic or non-therapeutic. In a particularly preferred embodiment of the current invention, the methods and uses are non-pharmaceutic, in particular, feeding applications.

Untreated manure of animals may have a detrimental environmental effect due to pathogenic bacteria and other ingredients, particularly with respect to the animals themselves and/or with respect to human beings coming in contact with the manure. This detrimental effect can be alleviated by either feeding the animals, or directly treating the manure or the bedding of the animals with the strains, compositions or preparations of the current invention. Therefore a further subject of the current invention is a method of controlling and/or avoiding detrimental environmental effects of manure or contaminated liquids, the method comprising the step of applying to manure, contaminated liquids, litter, a pit, or a manure pond at least one strain, one preparation and/or one composition according to the current invention. Preferably, the composition is applied in liquid form, for example by spraying, or as a powder, for example by strewing.

Detrimental bacteria may have a negative influence on the consistency of litter and in particular may affect a rather fluid or highly fluid litter, which might lead to foot pad lesions of poultry and which can be avoided by feeding the animals with the strains, compositions or preparations of the current invention. Therefore a further subject of the current invention is a method of controlling and/or improving the consistency of litter, in particular a method of ensuring a solid consistency of litter and/or a method of avoiding foot pad lesions, the method comprising the step of feeding animals, in particular poultry, at least one strain, one preparation and/or one composition according to the current invention.

The strains and preparations according to the invention can also be used for improving the quality of water. A further subject of the current invention is therefore also a method of controlling and/or improving the quality of water or aqueous solutions, in particular of drinking water and/or rearing water, comprising the step of applying to water or an aqueous solution at least one strain and/or at least one preparation and/or at least one composition of the current invention.

Further, the strains and preparations according to the invention can also be used for treating microbial diseases of plants. A further subject of the current invention is therefore also a method of treating and/or preventing microbial diseases of plants, in particular of cultivated plants, comprising the step of applying to the plants at least one strain and/or at least one preparation and/or at least one composition of the current invention. The application may be carried out in liquid form, such as by spraying, or in solid form, in particular as a powder.

In certain embodiments, where administering the strains of the present invention results an improvement of at least one of the features mentioned above is realized, wherein realization of the feature refers to an improvement of at least 1%, and more preferably of at least 3 or at least 5%, in comparison to an adequate negative control. As a negative control, averages known in the animal husbandry field may be used, but preferably animals which are subjected to the same treatment as the animals tested are used, but without administration of the strains and/or preparations of the current invention.

In particular, the strains, preparations and compositions of the current invention may be administered or fed to an animal in an amount effective to inhibit and/or decrease the growth of pathogenic bacteria in the animal gut. Such pathogenic bacteria include E. coli, Salmonella, Streptococcus, Rhodococcus, Enterococcus, and Clostridium. R elatedly, the methods of the present invention may be used to decrease the amount of pathogenic bacteria shed in animal feces. The methods of the present invention may also be used to maintain or increase the growth of beneficial bacteria, such as lactic acid bacteria, in the animal gut. By decreasing pathogenic bacteria and/or increasing or maintaining beneficial bacteria, the compositions of the present invention are able to maintain an overall healthy gut microflora.

Thus, a further subject of the current invention is a method of inhibiting and/or decreasing the growth of harmful or pathogenic bacteria and/or maintaining and/or increasing the growth of beneficial bacteria in an animal gut, wherein strains and/or preparations and/or compositions of the current invention are administered to animals and wherein the pathogenic bacteria are preferably selected from E. coli, Salmonella, Streptococcus, Rhodococcus, Enterococcus, and Clostridium.

In a preferred embodiment of the invention, the compositions of the present invention reduce the amount of at least one pathogenic bacterium by at least 0.5 log, and more preferably by at least 1 log, 2 log, or 3 log. In an embodiment, the compositions of the present invention can reduce the growth of pathogenic isolates by at least 75% in 12 hours, at least 80% in 12 hours, at least 85% in 12 hours, at least 90% in 12 hours, at least 95% in 12 hours, at least 75% in 20 hours, at least 80% in 20 hours, at least 85% in 20 hours, at least 90% in 20 hours, at least 95% in 20 hours.

Thus, a further subject of the current invention are the strains, preparations and compositions of the current invention for inhibiting and/or decreasing the growth of pathogenic bacteria and/or for maintaining and/or increasing the growth of beneficial bacteria in an animal gut, wherein the pathogenic bacteria are preferably selected from E. coli, Salmonella, Streptococcus, Rhodococcus, Enterococcus, and Clostridium.

A further subject of the current invention is therefore a therapeutic composition comprising the strains and/or compositions of the current invention as mentioned before. A preferred subject in this context is therefore a therapeutic composition for treatment and/or prevention of necrotic enteritis, in particular sub-clinical necrotic enteritis, in animals, preferably poultry, comprising the strains and/or compositions of the current invention as described herein.

Another preferred subject in this context is a therapeutic composition for treatment and/or prevention of bacterial enteritis, gangrenous dermatitis, colangiohepatitis, clostridiosis, diarrhea and/or foot pad dermatitis, in animals, preferably poultry, comprising the strains and/or compositions of the current invention as described herein.

A further subject of the current invention is therefore also the treatment and/or prevention of a disease, in particular of a gut disease, preferably of necrotic enteritis, in particular of sub-clinical necrotic enteritis, in poultry, wherein a strain and/or composition and/or preparation of the current invention is administered to an animal in need thereof.

A further subject of the current invention is also the treatment and/or prevention of a disease, preferably a disease of poultry, selected from bacterial enteritis, gangrenous dermatitis, colangiohepatitis, diarrhea and/or footpad dermatitis, wherein a strain and/or composition and/or preparation of the current invention is administered to an animal in need thereof.

In another embodiment, the Bacillus velezensis strain of the invention results in the production and secretion of enzymes that are beneficial to livestock by hydrolyzing molecules that can then be absorbed through the intestines. In one embodiment, the enzymes produced include, but are not limited to, one or more of aniylase, xylanase, protease, and lipase that break down one or more of starch, xylan, protein, and fats into glucose, xylose, amino acids, and free-fatty acids. In one embodiment, the enzymes produced include lipase and amylase in particular.

In another embodiment, the Bacillus velezensis strain of the invention results in the reduction of antibiotic use. In some embodiments, the Bacillus velezensis strain of the invention decreases the need for antibiotics by limiting the ability of hemolytic bacteria to grow. The reduction in antibiotic use may be compared to untreated animals or animals treated with another composition.

The strains and/or preparations and/or compositions of the current invention can be administered to animals in feed and/or drinking water over multiple days throughout the animal's life or during particular stages or portions of the animal's life. For example, the strains and/or compositions can be administered only in a starter diet or only in a finisher diet of farm animals.

In another aspect of the invention, the administration of the Bacillus velezensis strain PTA-127793 as described herein has been shown to provide numerous benefits to animal health and performance. Specifically, the methods of the invention are effective for improving body weight gain and feed conversion ratio. Furthermore, the administration of the strain can improve the overall health of the animal, which includes, but is not limited to, reducing mortality. The methods are also effective for the prophylactic prevention of diseases such as necrotic enteritis. In animals already experiencing a pathogenic infection, the administration of the strain can reduce the severity of associated symptoms. A significant commercial and health advantage is that the administration of the strain can decrease the overall need for therapeutic antibiotic use in an animal production system by improving the animal's baseline health and resistance to infection.

A particular subject of the current invention is also a method of enhancing the health of human beings and/or of improving the general physical condition of human beings and/or of increasing the disease resistance of human beings and/or of increasing the immune response of human beings and/or of establishing or maintaining a healthy gut microflora in human beings, wherein the strains and/or preparations of the current invention or the compositions of the current invention are administered to human beings.

A further subject of the current invention is the use of strains and/or preparations and/or compositions of the current invention for enhancing the health of human beings and/or for improving the general physical condition of human beings and/or for increasing the disease resistance of human beings and/or for increasing the immune response of human beings and/or for establishing or maintaining a healthy gut microflora in human beings, wherein the strains and/or preparations of the current invention or the compositions of the current invention, which comprise such strain(s), are administered to human beings.

The compositions of the present invention, in particular the feed, food and pharmaceutical compositions as well as the drinking or rearing water, preferably comprise the strain of the current invention and are administered to animals at a rate of about 1×10³ to about 2×10¹² CFU/g feed or mL water, in particular in a rate of about 1×10³ or about 1×10⁴ or about 1×10⁵ or about 1×10⁶ or about 1×10⁷ or about 1×10⁸ or about 1×10⁹ or about 1×10¹⁰ or about 1×10¹¹ or about 1×10¹² CEU/g feed or mL water, preferably in an amount of about 1×10³ to about 1×10¹⁴ CFU/g feed or mL water or about 1×10⁴ to about 1×10¹⁰ CF U/g feed or m L water, and more preferably in an amount of 1×10⁴ to 1×10⁷ CFU/g feed or mL water. In one embodiment, the strain is administered in an amount of 1×10⁵ CFU/g feed or mL water.

Correspondingly, preferred amounts of the strains and/or preparations of the current invention in the feed, food and water compositions of the current invention range preferably from 0.1 wt.-% to 10 wt.-%, more preferably from 0.2 wt.-% to 5 wt.-%, in particular from 0.3 wt.-% to 3 wt-%.

The methods of the present invention may be used for all kind of animals, in particular all kind of non-human and non-insect animals, more preferably all kind of vertebrates such as mammals, aquatic animals and birds.

Animals that may benefit from the current invention include but are not limited to farm animals, pets, exotic animals, zoo animals, aquatic animals, animals used for sports, recreation or work. Pets are preferably selected from dogs, cats, domestic birds and domestic exotic animals.

Aquatic animals are preferably selected from finfish and crustaceans which are preferably intended for human nutrition. These include, in particular, carp, tilapia, catfish, tuna, salmon, trout, barramundi, bream, perch, cod, shrimps, lobster, crabs, prawns and crayfish. Preferred types of salmon in this context are the Atlantic salmon, red salmon, masu salmon, king salmon, keta salmon, coho salmon, Danube salmon, Pacific salmon and pink salmon.

Further preferred aquatic animals are fanning fish which are subsequently processed to give fish meal or fish oil. In this connection, the fish are preferably herring, pollack, menhaden, anchovies, capelin or cod.

In a further preferred embodiment, the animals are farm animals, which may be raised for consumption or as food-producers, such as poultry, swine, equine (horses) and ruminants. The poultry may be selected from productive or domestic poultry, but also from fancy poultry or wild fowl. Preferred productive poultry in this context are chickens, turkeys, ducks and geese. The productive livestock in this context is preferably poultry optimized for producing young stock or poultry optimized for bearing meat. Preferred fancy poultry or wild fowl are peacocks, pheasants, partridges, chukkars, guinea fowl, quails, capercaillies, grouse, pigeons and swans, with quails being especially preferred. Further preferred poultry are ratites, in particular ostriches and emus, as well as parrots.

Ruminants according to the current invention are preferably selected from bovine (cattle), goats, sheep, and deer. In one embodiment, the compositions of this invention may be fed to preruminants to enhance their health and, in particular, to decrease the incidence of diarrhea in these animals Preruminants are ruminants, including calves, ranging in age from birth to about twelve weeks.

The compositions of the current invention may comprise at least one carrier or typical feed ingredients or combinations thereof. Suitable carriers are inert formulation ingredients added to improve recovery, efficacy, or physical properties and/or to aid in packaging and administration. Such carriers may be added individually or in combination. These carriers may be selected from anti-caking agents, flowability agents, anti-oxidation agents, bulking agents, surfactants, emulsifiers, binders, and/or protectants. Examples of useful carriers include polysaccharides (in particular starches, maltodextrins, methylcelluloses, gums, chitosan and/or inulins), protein sources (in particular skim-milk powder and/or sweet-whey powder), peptides, sugars (in particular lactose, trehalose, sucrose and/or dextrose), lipids (in particular lecithin, vegetable oils and/or mineral oils), salts (in particular sodium chloride, sodium carbonate, calcium carbonate, chalk, limestone, magnesium carbonate, sodium phosphate, calcium phosphate, magnesium phosphate and/or sodium citrate), and silicates (in particular clays, in particular beolite clay, amorphous silica, fumed/precipitated silicas, zeolites, Fuller's earth, baylith, clintpolite, montmorillonite, diatomaceous earth, talc, bentonites, and/or silicate salts like aluminium, magnesium and/or calcium silicate). Suitable carriers for animal feed additives are set forth in the American Feed Control Officials, Inc.'s Official Publication, which publishes annually See, for example Official Publication of American Feed Control Officials, Sharon Krebs, editor, 2006 edition, ISBN 1-878341-18-9. These carriers may include dextrin, silicon, bentonite, leonhardite, and zeolite. The carriers can be added after concentrating the fermentation broth and/or during and/or after drying. Preferred carriers according to the invention are selected from calcium carbonate, diatomaceous earth and vegetable oil.

A preferred embodiment of the current invention are concentrate compositions, in particular feed additive compositions, i.e. compositions suitable for preparing a feed composition, which comprise at least one strain of the current invention and at least one carrier, wherein the at least one strain is preferably comprised in an amount of 0.1 to 10 wt.-%, more preferably in an amount of 0.2 to 5 wt.-%, in particular in an amount of 0.3 to 3 wt.-%, and most preferably in an amount of 0.4 to 2.2 wt.-%, and the at least one carrier is preferably comprised of an amount of at least 90 wt. %, preferably in an amount of 90 to 99.9 wt.-%, more preferably in an amount of 95 to 99.8 wt.-%, in particular in an amount of 97 to 99.7 wt.-%, and most preferably in an amount of 97.8 to 99.6 wt.-%, and wherein the carrier consists preferably substantially of limestone, in particular of limestone with smaller parts of diatomaceous earth and/or vegetable oil.

These preferred compositions of the current invention, which contain stabilized strains, can be used for the preparation of feed and pharmaceutical compositions as well as drinking and rearing water which preferably comprise the strains according to the invention in an amount described or contemplated herein. In a preferred embodiment, 200 to 1000 grams of such a concentrate composition, in particular 250, 500 or 1000 grams of such a concentrate composition, are used per ton of feed, drinking or rearing water to provide compositions which can be used for feeding animals These concentrate compositions preferably comprise at least one strain of the current invention in an amount of 1×10⁹ to 2×10¹¹ CFU, in particular 2×10⁹ to 1×10¹¹ CFU, per g of the concentrate composition. Starting from these concentrate compositions, feed and food compositions can be prepared by mixing the concentrate compositions with typical feed or food ingredients, respectively.

Typical animal feed ingredients which may be contained in the compositions according to the invention and/or used in the preparation of feed compositions starting from concentrate compositions according to the invention include one or more of the following: proteins, carbohydrates, fats, further probiotics or microbes, prebiotics, enzymes, vitamins, immune modulators, milk replacers, minerals, amino acids, coccidiostats, acid-based products and/or medicines, such as antibiotics.

The compositions of the invention, such as an animal feed or animal feed additive, may further comprise an agriculturally acceptable carrier. The carrier may be selected from one or more of water, glycerol, ethylene glycol, 1,2-propylene glycol or 1,3-propylene glycol, sodium chloride, sodium benzoate, potassium sorbate, sodium sulfate, potassium sulfate, magnesium sulfate, sodium thiosulfate, calcium carbonate, sodium citrate, dextrin, maltodextrin, white oil, corn cob meal, rice bran, glucose, sucrose, sorbitol, lactose, wheat flour, wheat bran, corn gluten meal, starch, and cellulose. The composition may also comprise at least one additional enzyme, prebiotic, or probiotic. In some embodiments, the composition is provided as a supplement or milk replacer.

Carbohydrates containing components which may be used according to the invention are for example forage, roughage, wheat meal, sunflower meal or soya meal, and mixtures thereof.

Protein-containing components which may be used according to the invention are for example soya protein, pea protein, wheat gluten or corn gluten, and mixtures thereof.

Fat-containing components which may be used according to the invention are in particular oils, of both animal and plant origin, like vegetable oils, for example soya bean oil, rapeseed oil, sunflower seed oil, flaxseed oil or palm oil, fish oil, and mixtures thereof.

Protein-containing components which additionally contain fats which may be used according to the invention are for example fish meal, krill meal, bivalve meal, squid meal or shrimp shells, as well as combinations thereof.

Further probiotics (DFM) which may be used according to the invention in combination with the strains and preparations of the invention are preferably other strains of bacteria selected from the species Bacillus velezensis, Bacillus licheniformis, Bacillus lentus, Bacillus pumnilus, Bacillus laterosporus, Bacillus coagulans, Bacillus alevi, Bacillus cereus, Bacillus badius, Bacillus thurigiensis, Enterococcus faecium, and Pediococcus acidilactici. Other preferred bacteria are Bacillus velezensis PB6 (as described in U.S. Pat. No. 7,247,299 and deposited as ATCC Accession No. PTA-6737), which is sold by Kemin under the trademark CLOSTAT©, Bacillus velezensis D3, deposited under accession number PTA-127643 by Kemin Industries, Bacillus velezensis C-3102 (as described in U.S. Pat. No. 4,919,936 and deposited as FERM BP-1096 with the Fermentation Research Institute, Agency of Industrial Science and Technology, in Japan), sold by Calpis as CA LSPORIN®, Bacillus velezensis DSM 17299, as sold by Chr. Hansen under the trademark GalliPro®, a mixture of Bacillus velezensis DSM 17299 and Bacillus licheniformis DSM 17236, as sold by Chr. Hansen under the trademark GalliProTect®, a mixture of Bacillus licheniformis and Bacillus velezensis spores sold by Chr. Hansen under the trademark BIOPLUS2B®, or Bacillus coagulans strains as described in U.S. Pat. No. 6,849,256. Other non-Bacillus probiotics, such as Saccharomyces cerevisiae, Pichia pastoris, Aspeggillus niger, Aspeggillus oryzae, or Hansenula, may also be used in compositions of the present invention. In particular in food compositions further probiotics which are known to be useful to the human health may be used such as lactic acid producing bacteria, in particular lactobacilli, or Bifidobacteria. If said further probiotics are not formulated as part of the compositions of the present invention, they may be administered together (either at the same time or at different times) with the compositions of the present invention.

Prebiotics which may be used according to the invention are preferably oligosaccharides, in particular selected from galactooligosaccharides, sialyloligosaccharides, lactulose, lactosucrose, fructooligosaccharides, palatinose or isomaltose oligosaccharides, glycosyl sucrose, maltooligosaccharides, isomaltooligosaccharide, cyclodextrins, gentio oligosaccharides, soybean oligosaccharides, xylooligosaccharides, dextrans, pectins, polygalacturonic, rhamnogalacturonan, mannan, hemicellulose, arabinogalactan, arabinan, arabinoxylan, resistant starch, mehbiose, chitosan, agarose, inulin, tagatose, polydextrose, and alginate.

Enzymes which may be used in feed compositions according to the invention and which may further aid in the digestion of feed, are preferably selected from phytases (EC 3.1.3.8 or 3.1.3.26), xylanases (EC 3.2.1.8), galactanases (EC 3.2.1.89), galactosidases, in particular alpha-galactosidases (EC 3.2.1.22), proteases (EC 3.4.), phospholipases, in particular phospholipases Al (EC 31.1.32), A2 (EC 3.1.1.4.), C (EC 3.1.4.3), and D (EC 3.1.4-4), lysophospholipases (EC 3.1.1.5), amylases, in particular alpha-amylases (EC 3.2.1.1); lysozymes (EC 3.2.1.17), glucanases, in particular beta-glucanases (EC 3.2.1.4 or EC 3.2.1.6), glucoamylases, cellulases, pectinases, or any mixture thereof.

Examples of commercially available phytases include Bio-Feed™ Phytase (Novozymes), Ronozyme® P and HiPhos™ (DSM Nutritional Products), Natuphos™ (BASF), Finase® and Quantum® Blue (AB Enzymes), the Pbyzyme® XP (Verenium/DuPont) and Axtra® PHY (DuPont). Other preferred phytases include those described in e.g. WO 98/28408, WO 00/4′3503, and WO 03/066847.

Examples of commercially available xylanases include Ronozyme® WX and G2 (DSM Nutritional Products), Econase® XT and Barley (AB Vista), Xylathin® (Verenium) and Axtra® XB (Xylanase/beta-glucanase, DuPont). Examples of commercially available proteases include Ronozyme® ProAct (DSM Nutritional Products).

Vitamins which may be used according to the invention are for example vitamin A, vitamin D3, vitamin E, vitamin K, e.g., vitamin K3, vitamin B12, biotin, choline, vitamin V1, vitamin B2, vitamin B6, niacin, folic acid and panthothenate, e.g., Ca-D-panthothenate, or combinations thereof.

Immune modulators which may be used are for example antibodies, cytokines, spray-dried plasma, interleukins, or interferons, or combinations thereof.

Minerals which may be used according to the invention are for example boron, cobalt, chloride, chromium, copper, fluoride, iodine, iron, manganese, molybdenum, selenium, zinc, calcium, magnesium, potassium, or sodium, or combinations thereof. Amino acids which may be used according to the invention are for example lysine, alanine, threonine, methionine or tryptophan, or combinations thereof.

Thus, a further embodiment of the current invention is a method of preparing an animal feed composition comprising mixing at least one strain and/or at least one preparation and/or at least one concentrate composition of the current invention, in an amount effective to enhance animal health, with feed ingredients, such as proteins, lipids and/or carbohydrates, and optionally further beneficial substances, as mentioned above, to provide a feeding product. This method may comprise for example also a pelleting step.

Standard pelleting processes known to those of skill in the art may be used, including extrusion processing of dry or semi-moist feeds. Preferred pelleting temperatures are between about 65° C. and about 120° C. The composition of the current invention can withstand the pelleting process while losing less than 1 log.

In an embodiment, the compositions of the current invention may be sprayed onto animals or onto animal feed (including, but not limited to via micro-machine), mixed into feed at a feed mill, or mixed into the water supply. In an embodiment, a powdered forrmi of the bacterial strain is added from a microbin into a stream of water which is blended into a homogenous liquid mixture with other dry products. The liquid mixture is then applied to the feed by directly pumping it onto the feed in the feed mixer.

The strains and compositions of the present invention can be obtained by culturing the strains of the current invention according to methods well known in the art, including by using the media and other methods as described for example in U.S. Pat. No. 6,060,051, EP0287699 or US2014/0010792. Conventional large-scale microbial culture processes include submerged fermentation, solid state fermentation, or liquid surface culture.

Towards the end of fermentation, as nutrients are depleted, the cells of the strains begin the transition from growth phase to sporulation phase, such that the final product of fermentation is largely spores, metabolites and residual fermentation medium. Sporulation is part of the natural life cycle of these strains and is generally initiated by the cell in response to nutrient limitation. Fermentation is configured to obtain high levels of colony forming units of the Bacillus velezensis cells and to promote sporulation. The bacterial cells, spores and metabolites in culture media resulting from fermentation may be used directly or concentrated by conventional industrial methods, such as centrifugation, tangential-flow filtration, depth filtration, and evaporation. The concentrated fermentation broth may be washed, for example via a diafiltration process, to remove residual fermentation broth and metabolites.

The fermentation broth or broth concentrate can be dried with or without the addition of carriers using conventional drying processes or methods such as spray drying, freeze drying, tray drying fluidized-bed drying, drum drying, or evaporation. The resulting dry products may be further processed, such as by milling or granulation, to achieve a specific particle size or physical format. Carriers, as described above, may also be added post-drying.

Preparations of the strains of the current invention may be cell-free preparations or preparations containing cell debris or preparations containing a mixture of intact cells and cell debris. Cell-free preparations of the strains of the current invention can be obtained for example by centrifugation and/or filtration of fermentation broth. Depending on the technique used, these cell-free preparations may not be completely devoid of cells but may still comprise a smaller amount of cells. As the cells secrete compounds like metabolites, enzymes and/or peptides into the surrounding medium, the supernatant of the cells comprises a mixture of such compounds, in particular metabolites, enzymes and/or peptides, as secreted by the cells. Thus, in a preferred embodiment of the invention, the preparation of the strains is a supernatant of the fermentation broth.

Compositions comprising cell debris of the strains may be obtained by rupturing the cells applying techniques as known to those of skill in the art, for example by mechanical means or by applying high pressure. Depending on the degree of force applied, a composition comprising only ruptured cells or a composition comprising a mixture of cell debris and intact cells is obtained. Homogenization of the cells may be realized for example by utilizing a French cell press, sonicator, homogenizer, microfluidizer, ball mill, rod mill, pebble mill, bead mill, high pressure grinding roll, vertical sha Ft impactor, industrial blender, high shear mixer, paddle mixer, and/or polytron homogenizer. Suitable alternatives are enzymatic and/or chronical treatment of the cells.

Cell-free preparations of the current invention also comprise preparations which are obtained by first rupturing the cells by applying techniques as mentioned before and subsequently removing the cell debris and the remaining intact cells. Removing of the cell debris and remaining intact cells can be carried out in particular by centrifugation and/or filtration.

The preparations of the strains of the current invention may comprise as active compounds such as at least one metabolite, preferably a mixture of metabolites, as further described below, and/or at least one enzyme selected from proteases, xylanases and/or cellulases, and/or at least one peptide, and/or combinations thereof.

A preparation containing an effective mixture of metabolites can be obtained for example according to the methods set forth in U.S. Pat. No. 6,060,051. In particular, the preparation can be obtained by precipitating the metabolites in the preparations mentioned using organic solvents like ethyl acetate and subsequently redissolving of the precipitated metabolites in an appropriate solvent. The metabolites may subsequently be purified by size exclusion filtration that groups metabolites into different fractions based on molecular weight cut-off.

Preferably an effective amount of the strains and/or preparations and/or compositions of the current invention is used in the embodiments of the current invention. The term “effective amount” refers to an amount which affects at least one beneficial effect to an animal and/or to the environment, in comparison to an animal or environment that has not been administered the strains and/or preparations and/or compositions of the current invention, but that has otherwise been treated the same.

In case of therapeutic applications, preferably a therapeutic amount ofthe strains and/or preparations and/or compositions of the current invention is used. The term “therapeutic amount” refers to an amount sufficient to ameliorate, reverse or prevent a disease state in an animal. Optimal dosage levels for various animals can easily be determined by those skilled in the art, by evaluating, among other things, the composition's ability to (i) inhibit or reduce pathogenic bacteria in the gut at various doses, (ii) increase or maintain levels of beneficial bacteria and/or (iii) enhance animal health at various doses.

The invention will now be illustrated by the following non-limiting Examples.

### EXAMPLES

### Example 1: Enzymatic Activity of Bacillus velezensis N23

### Materials and Methods

Preparation of cell-free supernatant. One gram of Bacillus velezensis PB6, Bacillus velezensis FXA, Bacillus licheniformis G3, Bacillus velezensis D3, and ENTEROSURE® (a mix of lichiniformis and Bacillus velezensis) spores were transferred to individual 50 mL Falcon tubes. Saline (9 mL of 0.85%) was added to each tube and vortexed at high speed for three minutes. Each probiotic (1 mL) was transferred to 14 mL round bottom tubes containing saline (9 mL) to create a 100-fold dilution. Tubes were transferred to an 80° C. water bath for 10 minutes to kill vegetative cells. Tubes were subsequently transferred to chilled water to quickly cool the tubes. Each probiotic (1 mL) was transferred to individual flasks containing sterile Tryptic Soy Broth (TSB, Becton Dickinson, Sparks, MD) with 0.6% Yeast Extract (TSBYE, 100 mL). Flasks were incubated (New Brunswick Scientific Shaking Incubator, Model C24KC, Edison, NJ) overnight (~20-24 h) at 37° C. while shaking at 180 RPM under aerobic conditions. For N23 and C23, freezer stock (1 mL) was transferred to sterile TSBYE (100 mL) and incubated as stated above. Cultures (30 mL) of each overnight culture were transferred to 50 mL Falcon tubes and centrifuged with an Eppendorf 5804 R benchtop centrifuge (Eppendorf, Hamburg, Germany) for 10 minutes at 5000 RPM. Supernatant was decanted into 9 mL round bottom tubes and serially filtered with 0.45 and 0.22 μM syringe filters to make CFS, which was then assayed for enzyme activity.

Xylanase activity. Xylanase activity was assayed according to SOP LB-IV-20/094-E². Reagents are listed in Table 1.

TABLE 1
Xylanase activity assay reagents.

| Reagent | Formula | Manufacturer | Part No. | Lot |
|---|---|---|---|---|
| Sodium Phosphate | Na₂HPO₄•2H₂O | Fisher | S472-500 | 178955 |
| Dibasic Dihydrate | | | | |
| Potassium Phospahte | KH₂PO₄ | Fisher | P285-3 | 33426 |
| Monbasic | | | | |
| Citric Acid | C₆H₈O₇•H₂O | Fisher | A940-500 | 214888 |
| Beechwood Xylan | β-1,4, α-1,2-xylan | Sigma | X4252- | BCBH1776V |
| | | | 25G | |
| Sodium Carbonate | Na₂CO₃ | Fisher | S263-1 | 171716 |
| Anhydrous | | | | |
| Potassium Sodium | C₄H₄KNaO₆•4H₂O | Sigma | S2377- | 070M0108V |
| Tartrate Tetrahydrate | | | 1KG | |
| Sodium Bicarbonate | NaHCO₃ | Fisher | S233-500 | 170369 |
| Sodium Sulfate | Na₂SO₄ | Fisher | S421-1 | 195740 |
| Anhydrous | | | | |
| Copper (II) Sulfate | CuSO₄•5H₂O | Acros | 42361- | A008503201 |
| Pentahydrate | | | 5000 | |
| Sulfuric Acid, 95% | H₂SO₄ | Fisher | A300-212 | 168345 |
| Ammonium Molybdate | (NH₄)₆Mo₇O₂₄•4H₂O | Fisher | A674-500 | 175 |
| Tetrahydrate | | | | |
| Disodium Hydrogen | Na₂HASO₄•7H₂O | Alfa Aesar | A18725 | Y22F034 |
| Arsenate | | | | |
| Xylose | C₅H₁₀O₅ | Sigma | X-1500 | 61H0760 |

Standard stock solution (2000 μg/mL) was prepared by dissolving 0.2±0.01 g of xylose in 100 mL of deionized (DI) water. To prepare standard solutions, the stock solution was diluted 10-fold to make a 200 μg/mL solution. This solution was further diluted according to Table 2.

TABLE 2
Xylanase standard preparation scheme.

| μg xylose/ | Diluted solution 200 | DI water |
|---|---|---|
| mL | μg/mL | (mL) |
| 0 | — | 1 |
| 40 | 0.2 mL | 0.8 |
| 80 | 0.4 mL | 0.6 |
| 120 | 0.6 mL | 0.4 |
| 160 | 0.8 mL | 0.2 |

CFS (1 mL) was dissolved in 9 mL of 0.1M phosphate buffer pH 7.5 and then extracted by mixing for 15 minutes on a magnetic stirrer. Solutions were centrifuged at 4600 RPM for 5 minutes to obtain a clear enzyme solution. Extracted CFS solutions (1 mL) were transferred to 15 mL Falcon tubes in duplicate. Blank tubes were left empty. Xylan substrate (0.5%, 1 mL) was added to each tube at 15-second intervals, vortexed for 15 seconds, and then placed in a 50° C. water bath. Tubes were incubated for 15 minutes at 50° C. Next, Nelson's reagent C (1 mL) was added to each tube (including blanks) and vortexed for 15 seconds to stop the reaction. Extracted CFS was added to blank tubes and vortexed for another 15 seconds. Tubes, including blanks, were placed in boiling water for 20 minutes. Tubes were then cooled, and Nelson's color reagent (1 mL) was added to all tubes, and then tubes were briefly vortexed. DI water (5 mL) was added to each tube and vortexed for 5 minutes. Samples were centrifuged at 3000 RPM to obtain a clear solution. Samples (1 mL) were transferred to cuvettes and read at 540 nm on a QuickDrop Spectrophotometer (Molecular Devices, Holliston, MA).

Amylase activity. Amylase activity was determined with Phadebas starch tablets (PN: 1301 Lot 3P5012). CFS (200 μL) was transferred to 14 mL round bottom tubes in duplicate. DI water (200 μL) was added to blank tubes. DI water (4 mL) was added to each tube. Tubes were placed in a 37° C. water bath for 5 minutes. Using forceps, one Phadebas tablet was added to each tube, which were then vortexed and placed back in the water bath for 15 minutes. Sodium hydroxide (0.5 M, 1 mL) was added to each tube, and tubes were vortexed and centrifuged at 1300×g for 5 minutes. Supernatant was transferred to cuvettes and read at 620 nm with a QuickDrop Spectrophotometer. Optical density (OD) values of the blanks were subtracted from the samples. Amylase activity (U/L) was determined using the standard curve included with the Phadebas tablets.

Protease activity. Protease activity was determined with a Pierce Protease Assay Kit (PN: 23263 LotYL384777). Assay Buffer was prepared by dissolving the BupH Borate Buffer Pack in DI water (500 mL). Succinylated casein (1 vial) was dissolved in Assay Buffer (5 mL). Trypsin Stock Solution (50 mg/mL) was prepared by dissolving lyophilized TPCK Trypsin in DI water (1 mL). Trypsin Stock Solution was made into 50 μL aliquots and stored at −80° C. Trypsin Stock Solution was diluted 10-fold to 0.5 mg/mL and then diluted 2-fold to yield six standards. TNBSA (2,4,6-trinitrobenzene sulfonic acid) was prepared by adding TNBSA stock solution (100 μL) to 14.9 mL of Assay Buffer. In a 96-well clear-bottom microtiter plate, Succinylated Casein Solution (100 μL) was added to one set of wells. Assay Buffer (100 μL) was added to a duplicate set of wells to serve as blanks. Each CFS sample or standard (50 μL) was added to both the Succinylated Casein wells and the corresponding blank wells. The plate was incubated for 20 minutes at 37° C. TNBSA Working Solution (50 μL) was added to each well. and the plate was incubated for another 20 minutes at 37° C. Absorbance was measured at 450 nm with a Spectramax i3X microtiter plate reader (Molecular Devices, Sunnyvale, CA). Net absorbance was determined by subtracting the A450 of the blank from that of the corresponding Casein well. This ΔA450 value is the absorbance generated by the proteolytic activity of the CFS. Absorbance was plotted as ΔA450 against protease standard concentration to assess relative protease activity of the CFS.

Lipase Activity. Lipase activity was measured with an Abnova Lipase Assay Kit (PN: KA1654 Lot CEO1A11). The kit is based on an improved dimercaptopropanol tributyrate (BALB) method, in which SH groups formed from lipase cleavage of BALB react with 5,5′-dithio-bis(2-nitrobenzoic acid) (DTNB) to form a yellow colored product. The color intensity, measured at 412 nm, is proportionate to the enzyme activity in the sample. Working Reagent was prepared by combining Color Reagent (100 mg), Assay Buffer (280 μL), and BABL Reagent (160 μL). Calibrator and DI water (150 μL) were added to separate designated wells of a 96-well plate. CFS (10 μL) was added to appropriate wells followed by Working Reagent (140 μL). The plate was tapped to mix and read at 412 nm with a Spectramax i3X microtiter plate reader at 10 and 20 minutes. Lipase activity was determined by the below equation:

$$Activity = ( ( {OD}_{20 min} - {OD}_{10 min} ) / ( {OD}_{calibrator} - {OD}_{water} ) ) \times 735$$

where OD_{20 min} and OD_{10 min} are the OD_{412 nm} values of the sample at 20 and 10 minutes, respectively, and OD_{Calibrator} and OD_{water} are the OD_{412 nm} values of the calibrator and water at 20 minutes. The number “735” is the equivalent activity (U/L) of the calibrator under the assay conditions.

### Results

Lipase activity was highest in the PB6 CFS. Amylase and xylanase activity were highest in the CFS of N23, and protease activity was highest in the CFS from C23. Bacillus licheniformis (G3) CFS showed the lowest enzyme activity for all four enzymes. Complete results are in Table 3.

TABLE 3
Lipase, amylase, protease, and xylanase enzyme activity in cell-free
supernatant of D3, PB6, FXA, G3, C23, N23 and ENTEROSURE ®.
Mean ± Standard error (n = 3).

| | Lipase | Amylase | Protease | Xylanase |
|---|---|---|---|---|
| Probiotic | (U/L) | (U/L) | (μg/mL) | (U/mL) |
| D3 (Bacillus | 193 ± 0.01 | 41 ± 0.00 | 44 ± 0.00 | 54 ± 0.00 |
| velezensis) | | | | |
| PB6 (Bacillus | 483 ± 0.02 | 247 ± 0.02 | 39 ± 0.00 | 36 ± 0.00 |
| velezensis) | | | | |
| FXA (Bacillus | 240 ± 0.00 | 199 ± 0.00 | 41 ± 0.00 | 46 ± 0.00 |
| velezensis) | | | | |
| G3 (Bacillus | 74 ± 0.01 | 0.0 ± 0.00 | 29 ± 0.01 | 16 ± 0.01 |
| licheniformis) | | | | |
| ENT (Bacillus | 306 ± 0.01 | 128 ± 0.00 | 37 ± 0.01 | 20 ± 0.01 |
| velezensis and | | | | |
| Bacillus | | | | |
| licheniformis) | | | | |
| C23-2 (Bacillus | 396 ± 0.02 | 632 ± 0.00 | 47 ± 0.00 | 86 ± 0.00 |
| Velezensis) | | | | |
| N23 (Bacillus | 151 ± 0.00 | 716 ± 0.00 | 46 ± 0.00 | 108 ± 0.01 |
| velezensis) | | | | |

### Discussion

Enzymes produced and secreted by probiotics can be beneficial to livestock by hydrolyzing molecules that can then be absorbed through the intestines. Amylase, xylanase, protease, and lipase break down starch, xylan, protein, and fats into glucose, xylose, amino acids, and free-fatty acids. These probiotic-produced enzymes can improve feed efficiency and growth performance. Swiatkiewicz, S. et al. (2014). Feed enzymes, probiotic, or chitosan can improve the nutritional efficacy of broiler chicken diets containing a high level of distillers dried grains with solubles. Livestock Science 163 (2014) 110-119.

PB6 CFS showed the most lipase activity of the probiotics tested, but N23 and C23 were the top producers of amylase, protease, and xylanase. Bacillus velezensis D3 has shown strong in vitro inhibition but is not among the highest producers of lipase and amylase. It was the third-highest producer of protease and xylanse, behind only C23 and N23, however. While not the highest producer of all four enzymes tested, D3's in vitro efficacy and secretion of lipase, amylase, protease, and xylanase make it a well-rounded probiotic.

### Example 2: In Vitro Inhibition from Bacillus velezensis N23

To assess in vitro inhibition of select pathogens, Bacillus velezensis N23 was grown in Tryptic Soy Broth (TSB) containing 0.6% BD Yeast Extract (TSBYE, 100 mL) for 22-24 h at 37° C. under aerobic conditions with shaking (New Brunswick Scientific Shaking Incubator, Model C24KC, Edison, NJ) at 180 rpm. Cultures were centrifuged using an Eppendorf 5804 R benchtop centrifuge (Eppendorf, Hamburg, Germany) at 5,000 rpm for 10 min at room temp. To obtain cell-free supernatant (CFS), media was decanted into a 10 mL syringe and sterile filtered through a 0.22 μm syringe filter.

Pathogens were grown overnight in the media and conditions best suited for the specific organism. Pathogens grown aerobically were serially diluted first in sterile saline (0.85%) and then in sterile media to achieve a concentration of ~E6 CFU/mL. A 100 μL aliquot of challenge organism+a 100 μL aliquot of CFS were dispensed into appropriate wells of a 96-well microtiter plate. Positive controls consisted of 100 μL of the pathogen in media+100 μL of a 50:50 (v:v) mix of sterile saline and media, and a negative control was comprised of 100 μL of media (no organisms)+100 μL of sterile saline. A Spectramax i3X microtiter plate reader (Molecular Devices, Sunnyvale, CA) set to a 620 nm wavelength was used to measure the optical density (OD) of the suspension in each well. Plates were read kinetically every 2 h over 20 h. Temperature was maintained at 37° C. All results reflected the average OD measurements of four wells.

Well diffusion assays were used to evaluate the ability of N23 to inhibit Clostridium spp. CFS were individually prepared in the manner stated above. Agar best suited for the specific organism was seeded with 1 mL of E6 CFU/mL of inoculum from each Clostridial isolate. Alternately, the pathogen was swabbed onto the surface of the appropriate agar. The inoculated or swabbed agar plates were allowed to solidify or dry under laminar flow. Wells were made in the agar with a sterile pipette tip. CFS (50 μL) was added to wells. Sterile saline (50 μL) was added to one well (negative control). Chloramphenicol (Sensi Disk PN: 230733, Lot 2063283, 30 ug) was used as a positive control. Plates were left to sit at ambient for 30-60 min to facilitate absorption of the test fluid and then incubated in the upright position (agar down) overnight under anaerobic conditions at 37° C. An anaerobic environment was maintained using a BD anerobic gas generating pouch system (Benton Dickinson, Franklin Lakes, NJ). Zones of inhibition were measured to the nearest whole millimeter.

N23 inhibits growth of E. coli, Salmonella, Streptococcus, Rhodococcus, Enterococcus, and Clostridium. Partial and complete inhibition have been observed.

### Example 3: In Vitro Inhibition from Bacillus velezensis N23

Table 4 shows the kinetic read microtiter inhibition results from N23 cell-free supernatant.

TABLE 4
Percent inhibition; none (−), 1-25 (+), 26-50 (++), 51-75 (+++),
and 76-100 (++++).

| | | N23 | N23 |
|---|---|---|---|
| Isolate | ID | 12 h | 20 h |
| Hemolytic E. coli | 2022030295 | ++++ | ++++ |
| Hemolytic E. coli | 2021056941 | ++++ | +++ |
| Hemolytic E. coli | 2021036616 | +++ | +++ |
| Hemolytic E. coli | 2023048026 | +++ | ++ |
| Hemolytic E. coli | 2023033094 | ++++ | ++++ |
| Hemolytic E. coli | 2023009091 | ++++ | ++++ |
| Hemolytic E. coli | 2023031314 | ++++ | +++ |
| Hemolytic E. coli | 2023026033 | ++++ | ++++ |
| Hemolytic E. coli | 2023051522 | ++++ | ++++ |
| Hemolytic E. coli | 2023051522 | ++++ | ++++ |
| Hemolytic E. coli | 2021037480 | ++++ | ++++ |
| Hemolytic E. coli | 2023070969 | ++++ | ++++ |
| S. Typhimurium | M20-08867 | + | + |
| S. Litchfield | M21-05061-1 | + | + |
| S. Enterica | M20-24110-1-4 | ++++ | ++++ |
| S. Montevideo | M19-34817 | − | + |
| S. Enterica | M20-25381-1-7 | ++ | + |
| S. Panama | M21-05061-2 | + | + |
| S. Schwarzengrund | M21-28310 | + | + |
| S. aureus | ATCC 29213 | + | − |
| S. aureus | ATCC 25923 | ++++ | ++ |
| P. aeruginosa | ATCC 10145 | + | ++ |
| E. gallinarium | L23257007-6 | − | + |
| S. agalactiae | ATCC 12403 | +++ | − |
| S. Montevideo | ATCC 8387 | ++++ | ++++ |
| S. Typhimurium | ATCC 14028 | ++++ | ++++ |
| E. Coli | VDL-ISU 07-1690 | ++++ | ++++ |
| R. equi | VDL-ISU 45 | ++++ | ++++ |
| S. equi | VDL-ISU 43006 | ++++ | ++++ |
| S. Derby | ISU-SAL 0392-22 | ++++ | ++++ |
| E. cecorum | 21-87 | + | +++ |
| E. cecorum | 21-510 | ++++ | +++ |
| E. cecorum | 21-1684 | ++++ | ++++ |
| E. cecorum | 21-1685 | ++++ | ++++ |

As demonstrated, N23 showed strong in vitro inhibition of select common livestock pathogens. Of the 37 pathogens tested, N23 reduced growth of 24 (65%) and 20 (54%) isolates by 75-100% at 12 and 20 h, respectively.

### Example 4: Bacillus velezensis N23 Necrotic Enteritis Trial

Bacillus velezensis N23 is an intestinal health direct-fed microbial. N23 has shown superior in vitro performance against Salmonella, E. coli, and Clostridia when compared to CLOSTAT® (Bacillus subtillis PB6) and ENTEROSURE® (Bacillus PB6, FXA, and G3). The objective of the study was to evaluate the ability of N23 administered in feed to improve performance parameters in coccidiosis challenged broiler chickens at various doses.

CLOSTAT® was included as a positive control

This study was performed at Southern Poultry Feed and Research (SPFR) in Athens GA to evaluate the efficacy of the direct-fed microbial Bacillus velezensis N23 (N23) in mitigating necrotic enteritis in broiler chickens under commercial-like conditions. In this trial, N23 was administered at varying doses in feed. Cobb 500 male chicks were distributed across 70 pens, with each treatment replicated in 14 pens. Birds were raised on built-up litter, mimicking commercial U.S. poultry production, and all were vaccinated with a commercial coccidia vaccine. Feed intake, adjusted feed conversion ratio (FCR), weight gain, mortality, necrotic enteritis (NE) mortality, necrotic enteritis lesion scores, crypt depth and villi height, and microbiome data were analyzed. CLOSTAT® (Bacillus subtillis PB6) served as a positive control. N23 and PB6 feed intake, weight gain and adjusted FCR values did not significantly differ. However, N23 fed at 1E5 CFU/g had a four-point improvement in adjusted FCR and a 130 g greater weight gain when compared to PB6 fed at the same level during the 42-day trial. No differences were seen among treatments in microbiome alpha and beta diversity. PB6 and N23 improved necrotic enteritis lesion scores and necrotic enteritis mortality. Overall, N23 showed numerical improvements feed efficiency and weight when compared to PB6, and improved lesion scores and reduced mortality when compared to the challenged control.

### Materials and Methods

Treatments: Bacillus velezensis N23 Lot TexBio Batch 1 and CLOSTAT® (050245) lot 1808100487. Treatment feed consisted of a corn soy diet and was mixed at the SPFR feed mill and pelleted in a California Pellet Mill at 80° C. Diet (Table 5) and nutrient composition (Table 6) are below.

TABLE 5
Composition of the basal diet used for all treatments
in the necrotic enteritis challenge trial.

| | | Grower | |
|---|---|---|---|
| Ingredient | Starter | Percent | Finisher |
| corn | 59.00 | 63.96 | 68.21 |
| soybean meal | 34.86 | 29.88 | 25.85 |
| poultry fat | 1.83 | 2.35 | 2.51 |
| calcium carbonate | 1.14 | 1.05 | 0.96 |
| dicalcium phosphate. | 1.11 | 0.83 | 0.55 |
| dl-methionine | 0.43 | 0.38 | 0.37 |
| l-lysine | 0.43 | 0.41 | 0.43 |
| sodium bicarbonate | 0.24 | 0.22 | 0.22 |
| l-threonine 98.5 | 0.23 | 0.18 | 0.17 |
| salt, plain (nacl) | 0.22 | 0.24 | 0.24 |
| L-Arginine | 0.17 | 0.16 | 0.18 |
| L-Valine | 0.09 | 0.11 | 0.1 |
| L-Isoleucine | 0.08 | 0.06 | 0.06 |
| trace mineral¹ | 0.075 | 0.075 | 0.08 |
| vitamin premix | 0.05 | 0.05 | 0.05 |
| hostazym x | 0.03 | 0.03 | 0.03 |
| quantum blue | 0.01 | 0.01 | 0.01 |

¹Calcium minimum 3.20%, maximum 4.20%; Iron 2.63%; Magnesium 2.63%; Manganese 13.40%; Zinc 10.70%; Copper 4000 ppm; Iodine 1000 ppm; Selenium 400 ppm.
²Vitamin A 1,000,000 IU/lb; Vitamin D3 200,000 IU/lb; Vitamin E 2,000 IU/lb; Vitamin B-12 2.20 mg/lb; Riboflavin 800 mg/lb; Niacin 8,000 mg/lb; d-Pantothenic Acid 2,000 mg/lb; Choline 34,720 mg/lb; Menadione 132 mg/lb; Folic Acid 100 mg/lb; Thiamine 400 mg/lb; Pyridoxine 400 mg/lb; Biotin 20 mg/lb; Ethoxyquin 23,000 mg/lb.

TABLE 6
Nutrient composition of the basal diet used for all treatments
in the necrotic enteritis challenge trial. Digestible
(Dig) values are shown for individual amino acids.

| Nutrient | Starter | Grower | Finisher |
|---|---|---|---|
| dry matter | 85.41 | 85.18 | 84.94 |
| protein, crude | 21.74 | 19.64 | 18.06 |
| fat, crude | 4.12 | 4.74 | 4.98 |
| fiber, crude | 2.14 | 2.08 | 2.04 |
| calcium | 0.9 | 0.8 | 0.7 |
| phos. total | 0.58 | 0.5 | 0.44 |
| phos., available | 0.45 | 0.4 | 0.35 |
| m.e. poultry | 3,007 | 3,067 | 3,104 |
| methionine | 0.72 | 0.65 | 0.62 |
| lysine | 1.37 | 1.24 | 1.16 |
| tryptophan | 0.26 | 0.23 | 0.21 |
| threonine | 0.95 | 0.84 | 0.78 |
| sodium | 0.18 | 0.18 | 0.18 |
| potassium | 0.84 | 0.76 | 0.69 |
| chloride | 0.25 | 0.25 | 0.25 |
| dig methionine | 0.69 | 0.63 | 0.6 |
| dig cysteine | 0.24 | 0.22 | 0.21 |
| dig lysine | 1.26 | 1.14 | 1.07 |
| dig tryptophan | 0.26 | 0.23 | 0.21 |
| dig threonine | 0.86 | 0.75 | 0.7 |
| dig isoleucine | 0.86 | 0.76 | 0.7 |
| dig histidine | 0.46 | 0.43 | 0.4 |
| dig valine | 0.92 | 0.87 | 0.8 |
| dig leucine | 1.59 | 1.48 | 1.4 |
| dig arginine | 1.32 | 1.2 | 1.12 |
| dig phenylalanine | 0.92 | 0.84 | 0.78 |
| dig TSAA | 0.95 | 0.87 | 0.83 |

¹TSAA = total sulfur amino acids

Experimental direct-fed microbial. The colony forming units per gram (CFU/g) in each treatment provided to the test facility is shown in Table 7.

TABLE 7
CFU/g counts of direct-fed microbial treatments prior
to addition to feed (n = 3).

| | Direct-Fed | Target | Actual Counts |
|---|---|---|---|
| Treatment | Microbial | (CFU/g) | (CFU/g) |
| 3 | PB6 | 2E+08 | 5.05E8 |
| 4 | N23 | 2E+08 | 2.95E8 |
| 5 | N23 | 2E+10 | 3.55E9 |

Treatment 3 (CLOSTAT®) was prepared at 5.1 E8 CFU/g by combining 6.0 g of CLOSTAT® concentrate (1E11 CFU/g; 050245 lot 1808100487) and 1994 g of calcium carbonate fre-flo (RM01029 lot 2407108576). Treatments 4 and 5 were prepared at 3.0E8 and 3.6E9 CFU/g by combining 15.0 and 150.0 g of N23 concentrate (4E10 CFU/g; TexBio Batch 1) and 1985 and 1850 g of calcium carbonate fre-flo (RM01029 lot 2407108576), respectively. Each experimental treatment was assayed according to KANA method AMPD M-16. In brief, 10-fold dilutions in 0.85% saline (saline) were prepared, vortexed, and further diluted 10-fold to 10⁻⁸-10⁻⁹ with saline. The second dilutions (10⁻²) were heated to 80° C. for 10 min and immediately cooled in a cold-water bath for two minutes. Dilutions (1 mL of 10⁻⁶, 10⁻⁷, and 10⁻⁸) were plated on Neogen aerobic count (AC) petrifilms (Lansing, MI), and incubated aerobically overnight at 37° C.

Feed and water. Feed formulations for this study consisted of unmedicated commercial-type broiler starter diets compounded with commonly used United States feedstuffs representative of local formulations calculated to meet or exceed National Research Council (NRC) standards. No antibiotics were added to any feed. Treatments were prepared from basal feed. The basal diet and water were provided ad libitum from day 0 through 42 of the study. Broiler diets were fed as crumbles (starter feed) or as pellets (grower and finisher). All feed was administered by pen. Starter feed was issued and fed from day 0 to 21. On d21, unconsumed starter feed was weighed and discarded. Grower feed was issued on day 21 and fed until day 35. On day 35, unconsumed grower feed was weighed and discarded. Finisher feed was issued on day 35 and fed until day 42. On day 42, unconsumed finisher feed was weighed and discarded.

Feed sample analysis. Post-pelleted feed samples were tested by KANA R and D to verify the targeted CFU/g in feed. The target treatment level for CLOSTAT® was 1E5 CFU/g while the target inclusion level for N23 was 1E5 (treatment group 4) and 1E6 (treatment group 5) CFU/g. Feed samples were tested according to method AMPD M-14. Briefly, triplicate 10-fold (5.00 g treatment: 45.00 mL of 0.85% saline) dilutions were prepared, stomached in a Seward Stomacher 80 (Seward, Islandia, NY) for 30 seconds at medium speed, and further diluted 10-fold with saline. Dilutions (10⁻⁴ and 10⁻⁵) were plated in duplicate on Neogen AC films, and incubated aerobically overnight at 37° C.

Experimental facility. This study was designed to mimic commercial conditions as close as possible. The floor pen unit was GM Unit #1 located at 2011 Brock Road, Athens, GA. The experimental house of GM Unit #1 is divided into pens of equal size, arranged along a central aisle. Birds were placed in 70 pens each having an area of 4×10=40 ft². The initial stocking density, after subtracting for equipment, was ~0.91 ft²/bird or 44 birds per pen. There were 14 pens and 616 birds per treatment. All pens had approximately 4 inches of built-up litter with a coating of fresh pine shavings. Poultry production in the United States routinely uses built-up litter (litter from previous trials). Each pen had five-feet-high side walls with the bottom 1.5 feet being solid wood to prevent bird migration.

Design of the necrotic enteritis challenge trial. Day of hatch Cobb 500 male chicks were obtained from Cobb Vantress hatchery, Cleveland, GA; 3080 chicks were allocated to the study. Birds were handled according to Institutional Animal Care and Use Committee (IACUC) guidelines and monitored by a Southern Poultry Feed and Research (SPFR) veterinarian. Accountabilities of all test animals and any extra birds were recorded on the animal disposition form. The breeder flock history and vaccination record at the hatchery were recorded. Only healthy appearing chicks were used in the study. All birds were spray vaccinated with a commercial coccidia vaccine (Coccivac-B52) at the recommended use level prior to placement in pens. At study initiation, 44 chicks were allocated to each treatment pen by blocks. Chicks were allocated at random by block to each pen and carefully counted to assure the correct numbers of birds were placed into each experimental pen. No birds were replaced during the study. Bird weights (kg) by pen were recorded at study initiation (day 0) and on days 14, 21, 35, and 42. Bird weights (kg) by pen were recorded at study initiation (day 0) and on days 14, 21, 35, and 42. Experimental treatment groups, day of cocci challenge, and day of Clostridia perfringens challenge in shown in Table 8.

TABLE 8
Feed treatment types, challenge, and inclusion levels.

| | | Coccidia | Clostridium | |
|---|---|---|---|---|
| | | Challenge | perfringens | Pens/ |
| No. | Treatment | (Day) | (Day) | Treatment |
| 1 | Unchallenged | 14 | No | 14 |
| | Control | | | |
| 2 | Challenged | 14 | 19, 20, and 21 | 14 |
| | Control | | | |
| 3 | PB6 (1E5 CFU/g) | 14 | 19, 20, and 21 | 14 |
| 4 | N23 (1E5 | 14 | 19, 20, and 21 | 14 |
| | CFU/g) | | | |
| 5 | N23 (1E6 | 14 | 19, 20, and 21 | 14 |
| | CFU/g) | | | |
| | | | Total Pens | 70 |

Disease induction. On day 14, all birds, including treatment 1 (unchallenged control), were orally inoculated with ~5,000 oocysts of E. maxima per procedures described in a SPFR SOP. The SOP is proprietary and was not shared. Starting day 19, all birds, except treatment 1, were given 1 mL of a broth culture of Clostridia perfringens (CP) containing ~1E8 CFU/mL. Birds in the unchallenged control group were given sterile water (1 mL). The CP is an isolate from a clinical case of necrotic enteritis. It is both alpha-toxin and net beta-toxin positive. The birds were administered a fresh broth culture once daily for three days (days 19, 20, and 21).

Monitoring. All birds were monitored for general flock condition. In addition, temperature, lighting, water, feed, litter condition, and any unanticipated house conditions/events were noted. Findings were documented twice daily during the regular working hours with one observation recorded on the final day of the study.

Performance and mortality parameters. The birds and feed were weighed by pen on d0, 14, 21, 35, and 42. Means for pen weight gain, feed consumption, and FCR were then calculated (d0-14, 0-21, 0-35, 0-42, 14-21, 21-35, and 35-42). FCR was adjusted to account for mortality occurring during the study.

Necrotic enteritis intestinal lesion score. On day 21 and 35, three birds from each pen were euthanized, weighed, and examined for the degree of presence of NE lesions. The scoring was based on a 0 to 3 score, with 0 being normal and 3 being the most severe.
Lesion score 0=Normal
Lesion score 1=Slight mucus covering small intestine
Lesion score 2=Necrotic small intestine mucosa
Lesion score 3=Sloughed and bloody small intestine mucosa and contents

Statistical analysis. SPFR analyzed trial data with STATIX, using least-squared differences for analysis of means (p<0.05). The data were further evaluated by Kemin R and D using the JMP software package (version 18.0.0), using analysis of variance (ANOVA) with comparison of means using Tukey t-test (p<0.05).

Microbiome samples. Ceca from ten birds in treatment groups 1, 2, 3, and 5, were collected on day 42. Cecal contents were squeezed into suitable flip top tubes, quickly frozen on dry ice, and shipped from SPFR to Kemin on dry ice. Upon receipt, samples were stored at −80° C. until being processed. Cecal contents (~200 mg) were purified using a Qiagen Power Soil© Pro Kit (Ref.: 4706 Lot 175043714, Hilden Germany). Samples were purified per instructions in Kemin's Microbiome Sampling Protocol. Samples were eluted in Cytiva HyPure™ Molecular Biology Grade nuclease-free water (80 μL, Hyclone Cat. No. SH30538.02 LotAK30774141). Purified double stranded DNA (dsDNA; 5 μL) was combined with Qubit™ 1×dsDNA BR Working Solution (195 uL, Ref.: Q33260 Lot 2725319) and briefly vortexed. Samples were quantified with a Qubit 4 Fluorometer (Invitrogen PN: Q33226 SN: 23226240100050). Purified DNA was shipped to KAE for Oxford Nanopore Technology (ONT) sequencing. Additionally, samples (8 μL) were combined by group for metagenomic sequencing. ONT sequencing was performed by PathoSense, Ghent University, Ghent Belgium.

Villi testing: On day 42, one bird per pen (all pens; 70 birds) was sacrificed. The duodenum, jejunum and ileum were collected. Samples were rinsed with distilled water and preserved in neutral buffered formalin (10%) solution. Samples were sent to UGA where villus height and the crypt depth measurements were determined.

### Results

Feed was provided as crumbles (starter) or pellets (grower/finisher), and post-pelleted feed samples were tested to verify the level of N23 and PB6 in feed. Treatments 1 and 2 did not include a direct-fed microbial and were analyzed for background aerobic counts only. Treatment 3 contained PB6 at a target level of 1E5 CFU/g. Treatments 4 and 5 contained N23 at target levels of 1E5 and 1E6 CFU/g of feed, respectively. Expected treatment counts were calculated by multiplying the treatment concentration (CFU/g) by the treatment rate (g/1000 kg) and dividing by the amount of feed treated. Expected counts were higher than target treatment counts due to the treatment counts (CFU/g) being higher than target (Table 7). Over formulating treatments is by design due to expected losses of direct-fed microbials during the pelleting process. Basal feed AC background counts from treatments 1 and 2 ranged from 8.0E3 to 3.4E4 CFU/g. Treatment 3 feed counts were in line with expected values of 1E5 CFU/g PB6 for all feed mixes (starter, grower finisher). Treatment 4 and 5 counts were 0.25 to 0.5 log below the calculated expected N23 counts of 1E5 and 1E6 CFU/g, respectively, for each feed mix. N23 treatments were over formulated to a lesser extent than PB6 (Table 7) which likely led to lower recoveries from feed. See Table 9.

TABLE 9
Counts (CFU/g) in post-pelleted feed samples. (n = 3)

| | | | | | Actual Feed |
|---|---|---|---|---|---|
| | | Direct-Fed | Target | Expected | Counts |
| Treatment | Feed | Microbial | (CFU/g) | (CFU/g) | (CFU/g) |
| 1 and 2 | Starter | N/A | N/A | N/A | 8.0E3 |
| 3 | Starter | PB6 | 1E5 | 2.53E5 | 1.17E5 |
| 4 | Starter | N23 | 1E5 | 1.48E5 | 7.52E4 |
| 5 | Starter | N23 | 1E6 | 1.78E6 | 6.33E5 |
| 1 and 2 | Grower | N/A | N/A | N/A | 3.40E4 |
| 3 | Grower | PB6 | 1E5 | 2.53E5 | 1.04E5 |
| 4 | Grower | N23 | 1E5 | 1.48E5 | 7.48E4 |
| 5 | Grower | N23 | 1E6 | 1.78E6 | 5.38E5 |
| 1 and 2 | Finisher | N/A | N/A | N/A | 1.73E4 |
| 3 | Finisher | PB6 | 1E5 | 2.53E5 | 1.15E5 |
| 4 | Finisher | N23 | 1E5 | 1.48E5 | 5.60E4 |
| 5 | Finisher | N23 | 1E6 | 1.78E6 | 6.40E5 |

Overall mortality was highest in the unchallenged control group at 19.6% but did not significantly differ (p<0.05) from the other treatment groups (Table 10).

TABLE 10
Average percent mortality of birds for days 0-42. Mean ± SE.

| | | Percent |
|---|---|---|
| No. | Treatment | Mortality ± SE |
| 1 | Unchallenged Control | 19.6 ± 1.5 |
| 2 | Challenged Control | 17.0 ± 1.5 |
| 3 | PB6 (1E5 CFU/g) | 17.2 ± 1.5 |
| 4 | N23 (1E5 CFU/g) | 16.1 ± 1.5 |
| 5 | N23 (1E6 CFU/g) | 14.4 ± 1.5 |

SE: standard error

All birds found dead were necropsied. NE mortality (d0-42), or death attributed to necrotic enteritis lesions in the small intestines, was highest in the challenged control (no additive, CP) group at a rate of 5.4% and was significantly higher than all other groups. Groups 1, 3, 4, and 5 had NE mortality rates of 0.2, 2.6, 2.4 and 1.9%, respectively, and did not significantly differ. NE mortality results are summarized in Table 11.

TABLE 11
Percent necrotic enteritis mortality (d0-42) of broilers in
all treatment groups.

| | | Percent NE |
|---|---|---|
| No. | Treatment | mortality ± SE |
| 1 | Unchallenged Control | 0.2 ± 0.6^{b} |
| 2 | Challenged Control | 5.4 ± 0.6ª |
| 3 | PB6 (1E5 CFU/g) | 2.6 ± 0.6^{b} |
| 4 | N23 (1E5 CFU/g) | 2.4 ± 0.6^{b} |
| 5 | N23 (1E6 CFU/g) | 1.9 ± 0.6^{b} |

NE: Necrotic enteritis;
SE: standard error;
CP: C. perfringens
All groups' sans treatment 1 were challenged with C. perfringens on d19, 20 and 21.
Mean ± SE.
Values not connected the by same letter^{a-b} are significantly different (p < 0.05).

On day 21 and day 35, three birds from each pen were sacrificed, weighed, and examined for the degree of NE lesions. Day 21 NE lesion scores were highest in the challenged control group with a score of 1.5 and was significantly higher than all other groups. Groups 3, 4, and 5 had day 21 lesion scores of 1.2, 1.0, and 1.0 respectively, and did not significantly differ but were significantly higher than the unchallenged control value of 0.0. Necrotic enteritis day 35 lesion scores were highest in the challenged control group with a score of 2.3 and was significantly higher than group 4. Groups 3, 4, and 5 had day 35 lesion scores of 2.0, 1.9, and 2.0 respectively, and did not significantly differ but were significantly higher than the unchallenged control value of 0.0. NE lesion scores are summarized in Table 12.

TABLE 12
Average day 21 and 35 NE lesion scores of broilers in all treatment groups.

| | Day 21 average NE | Day 35 average NE |
|---|---|---|
| Treatment | lesion score ± SE | lesion score ± SE |
| Unchallenged Control | 0.0 ± 0.1^{a} | 0.0 ± 0.1^{c} |
| Challenged Control | 1.5 ± 0.1^{c} | 2.3 ± 0.1ª |
| PB6 (1E5 CFU/g) | 1.2 ± 0.1^{ab} | 2.0 ± 0.1^{ab} |
| N23 (1E5 CFU/g) | 1.0 ± 0.1^{b} | 1.9 ± 0.1^{b} |
| N23 (1E6 CFU/g) | 1.0 ± 0.1^{b} | 2.0 ± 0.1^{ab} |

NE: Necrotic enteritis;
SE: standard error;
CP: C. perfringens
All groups' sans treatment 1 were challenged with C. perfringens on d19, 20, and 21.
Mean ± SE.
Values not connected the by same letter^{a-c} are significantly different (p < 0.05).

No differences in feed intake were seen for days 0-14, 0-21, and 14-21. The unchallenged control group had significantly lower feed intake than groups 4 and 5 on days 0-35 and 0-42. No differences were seen among the treated groups. The unchallenged control was significantly lower than groups 3, 4, and 5 for days 21-35 and was also lower than group 4 on days 35-42. Feed intake results are in Table 13.

TABLE 13
Feed intake for day 0-14, 0-21, 0-35, 0-42, 14-21, 21-35, and 35-42 of broilers in all treatment groups.
Feed Intake (g)

| | | Days | | | | | | |
|---|---|---|---|---|---|---|---|---|
| Treatment No. | Treatment | 0-14 | 0-21 | 0-35 | 0-42 | 14-21 | 21-35 | 35-42 |
| 1 | Unchallenged Control | 22.7 | 44.7 | 111.5^{b} | 141.7^{b} | 22.0 | 66.8^{b} | 30.2^{b} |
| 2 | Challenged Control | 22.3 | 44.3 | 114.6^{ab} | 148.9^{ab} | 22.0 | 70.3^{ab} | 34.3^{ab} |
| 3 | PB6 ( E5 CFU/g) | 22.2 | 45.3 | 116.7^{ab} | 150.1^{ab} | 23.0 | 71.5^{a} | 33.4^{ab} |
| 4 | N23 (1E5 CFU/g) | 22.3 | 45.0 | 118.2^{a} | 154.2^{a} | 22.7 | 73.2^{a} | 36.1^{a} |
| 5 | N23 (1E6 CFU/g) | 23.3 | 46.0 | 117.4^{a} | 151.8^{a} | 22.8 | 71.4^{a} | 34.5^{ab} |
| | Standard Error | 0.3 | 0.6 | 1.4 | 2.3 | 0.4 | 1.1 | 1.3 |

All treatments sans treatment 1 were challenged with C. perfringens on d19, 20, and 21.
Mean ± standard error.
Values not connected by the same letters^{a-b} indicate significant differences in means (P < 0.05).

No differences in adjusted FCR were seen for days 0-14 and 35-42 and no differences were seen between treatments for any timepoint. For days 0-35, 0-42 and 21-35 the challenged control adjusted FCR was significantly worse than all other groups. For days 14-21 the adjusted FCR was significantly better in group 1 than all other groups and group 2 was significantly worse than groups 4 and 5. Adjusted FCR results are in Table 14.

TABLE 14
Adjusted FCRfor day 0-14, 0-21, 0-35, 0-42, 14-21, 21-35, and 35-42 of broilers in all treatment groups.
Adjusted FCR

| | | Days | | | | | | |
|---|---|---|---|---|---|---|---|---|
| Treatment No. | Treatment | 0-14 | 0-21 | 0-35 | 0-42 | 14-21 | 21-35 | 35-42 |
| 1 | Unchallenged Control | 1.26 | 1.31^{c} | 1.48^{b} | 1.62^{b} | 1.37^{c} | 1.63^{b} | 2.54 |
| 2 | Challenged Control | 1.25 | 1.40^{a} | 1.61^{a} | 1.78^{a} | 1.60^{a} | 1.80^{a} | 2.81 |
| 3 | PB6 (1E5 CFU/g) | 1.21 | 1.35^{b} | 1.52^{b} | 1.67^{b} | 1.52^{ab} | 1.65^{b} | 2.68 |
| 4 | N23 (1E5 CFU/g) | 1.21 | 1.33^{bc} | 1.51^{b} | 1.63^{b} | 1.48^{b} | 1.66^{b} | 2.28 |
| 5 | N23 (1E6 CFU/g) | 1.22 | 1.33^{bc} | 1.49^{b} | 1.63^{b} | 1.46^{b} | 1.63^{b} | 2.58 |
| | Standard Error | 0.02 | 0.01 | 0.01 | 0.01 | 0.02 | 0.03 | 0.16 |

All treatments sans treatment 1 were challenged with C. perfringens on d19, 20, and 21.
Mean ± standard error.
Values not connected by the same letters^{a-b} indicate significant differences in means (P < 0.05).

There were no differences in body weight gain for days 0-14 and no differences were seen among treatments for any time period. For days 0-21 weight gain was significantly higher in group 1 than all other groups and for days 0-35, 0-42, and 21-35 weight gain was significantly lower in group 2 than all other groups. For days 14-21 weight gain in group 1 was higher than all other groups and group 2 was lower. For days 35-42 weight gain was highest in group 4 and was significantly higher than the weight gained in group 2. Weight gain results are in Table 15.

TABLE 15
Weight gain for day 0-14, 0-21, 0-35, 0-42, 14-21, 21-35, and 35-42 of broilers in all treatment groups.
Weight Gain (kg)

| | | Days | | | | | | |
|---|---|---|---|---|---|---|---|---|
| Treatment No. | Treatment | 0-14 | 0-21 | 0-35 | 0-42 | 14-21 | 21-35 | 35-42 |
| 1 | Unchallenged Control | 0.41 | 0.75^{b} | 1.89^{a} | 2.53^{a} | 0.34^{a} | 1.14^{a} | 0.64^{ab} |
| 2 | Challenged Control | 0.41 | 0.68^{a} | 1.70^{b} | 2.30^{b} | 0.27^{c} | 1.02^{b} | 0.60^{b} |
| 3 | PB6 (1E5 CFU/g) | 0.42 | 0.72^{a} | 1.86^{a} | 2.51^{a} | 0.30^{b} | 1.14ª | 0.64^{ab} |
| 4 | N23 (1E5 CFU/g) | 0.42 | 0.73^{a} | 1.90^{a} | 2.64^{a} | 0.31^{b} | 1.17^{a} | 0.74^{a} |
| 5 | N23 (1E6 CFU/g) | 0.43 | 0.74ª | 1.87^{a} | 2.54^{a} | 0.30^{b} | 1.13^{a} | 0.67^{ab} |
| | Standard Error | 0.01 | 0.01 | 0.02 | 0.04 | 0.01 | 0.02 | 0.03 |

All treatments sans treatment 1 were challenged with C. perfringens on d19, 20, and 21.
Mean ± standard error.
Values not connected by the same letters^{a-b} indicate significant differences in means (P < 0.05).

Microbiome alpha diversity (α-diversity) is defined as the species diversity within each sample. Here, alpha diversity was estimated as the Shannon and Chao1 diversity. To assess differences in the microbial diversity between samples, a Kruskal-Wallis test was used evaluate whether the Shannon and Chao1 diversities were significantly different between treatments. The results of this test indicated no significant differences in either Shannon diversity or Chao1 diversity. A pairwise comparison between treatments was also performed using the Wilcoxon rank-sum test. Results are in Table 16.

TABLE 16
Results of the Wilcoxon rank-sum test comparing the Shannon
and Chao1 diversity pair-wise. P-values are adjusted.

| Group comparison | p-value Shannon | p-value Chao1 |
|---|---|---|
| T_01 vs T_02 | 1.00 | 1.00 |
| T_01 vs T_03 | 1.00 | 1.00 |
| T_01 vs T_04 | 1.00 | 0.26 |
| T_02 vs T_03 | 1.00 | 1.00 |
| T_02 vs T_04 | 0.31 | 0.26 |
| T_03 vs T_04 | 0.45 | 0.66 |

A beta diversity (β-diversity) analysis was performed to evaluate the dissimilarity in bacterial communities between samples. Here, the Bray-Curtis dissimilarity index was used to determine the degree of community differentiation between all samples within a data set. The community dissimilarity between the treatments was statistically tested for significant differences using a Permutational ANOVA (PERMANOVA) test with 9,999 permutations. The PERMANOVA test indicated no significant difference in microbial composition between the treatments. Pairwise PERMANOVA tests were performed for pairwise comparisons of the treatments (Table 17). Briefly, no significant differences have been detected in pairwise comparison.

TABLE 17
Results of the PERMANOVA statistical test with 9,999 permutations.

| | Degrees | | | | |
|---|---|---|---|---|---|
| Group | of | Sum of | | | |
| comparison | freedom | squares | R2 | F statistic | p value |
| T_01 vs T_02 | 1 | 0.0621 | 0.0479 | 0.9063 | 0.6598 |
| T_01 vs T_03 | 1 | 0.088 | 0.0589 | 1.1271 | 0.5545 |
| T_01 vs T_04 | 1 | 0.0968 | 0.0551 | 1.0495 | 0.5545 |
| T_02 vs T_03 | 1 | 0.1032 | 0.0711 | 1.3777 | 0.3222 |
| T_02 vs T_04 | 1 | 0.1236 | 0.0716 | 1.3886 | 0.3222 |
| T_03 vs T_04 | 1 | 0.0805 | 0.0434 | 0.8169 | 0.6598 |

Average villi height in duodenum samples was highest in intestinal samples obtained from birds fed PB6 and were significantly higher than the other treatments. Jejunum villi height was highest in intestinal samples obtained from N23 (1E6 CFU/g) and was significantly higher than PB6 and unchallenged control intestinal samples. Ileum villi height was highest in intestinal samples obtained from birds in the challenged control and was higher than all other treatment groups. Results are in Table 18.

TABLE 18
Average villi height (μM) of the duodenum, jejunum and ilium
sections of the small intestine obtained on day 42.

| | | Average Villi Height (μM) | | |
|---|---|---|---|---|
| | | Duodenum | Jejunum | Ilium |
| 1 | Unchallenged | 2009.4^{b} | 1031.7^{c} | 828.1^{b} |
| | Control | | | |
| 2 | Challenged | 1997.7^{b} | 1501.2^{ab} | 1178.3^{a} |
| | Control | | | |
| 3 | PB6 (1E5 CFU/g) | 3124.1^{a} | 1447.9^{b} | 943.7^{b} |
| 4 | N23 (1E5 CFU/g) | 1893.3^{b} | 1568.2^{ab} | 862.3^{b} |
| 5 | N23 (1E6 CFU/g) | 2031.3^{b} | 1800.4ª | 808.4^{b} |

Values not connected by the same letter^{(a-b)} are significantly different.

Crypt depths of the duodenum was deepest in samples obtained from birds in the unchallenged control group and were significantly deeper than the challenged control and both N23 treatment groups. Crypt depth in duodenal tissue was the shallowest in N23 (1E5 CFU/g) and was shallower than all other groups except N23 (1E6 CFU/g). Crypt depth of jejunum samples was deepest in intestinal samples obtained from the unchallenged control and were deeper than jejunum samples obtained from the groups fed PB6 and N23 (1E5 CFU/g). Jejunum samples obtained from birds in the N23 (1E5 CFU/g) had the shallowest crypts and were shallower than all other groups sans samples from birds fed PB6. Birds in the challenged control had the deepest ilium crypts and were significantly deeper than all other treatment groups. Results are shown in Table 19.

TABLE 19
Average crypt depth (μM) of the duodenum, jejunum and
ilium sections of the small intestine obtained on day 42.

| | | Average Crypt Depth (μM) | | |
|---|---|---|---|---|
| | | Duodenum | Jejunum | Ilium |
| 1 | Unchallenged | 166.89^{a} | 138.54^{a} | 90.27^{b} |
| | Control | | | |
| 2 | Challenged | 118.05^{bc} | 127.69^{a} | 153.04^{a} |
| | Control | | | |
| 3 | PB6 (1E5 CFU/g) | 141.89^{ab} | 98.19^{bc} | 86.06^{b} |
| 4 | N23 (1E5 CFU/g) | 89.10^{d} | 85.74^{c} | 78.08^{b} |
| 5 | N23 (1E6 CFU/g) | 106.19^{cd} | 114.82^{ab} | 75.99^{b} |

Values not connected by the same letter^{(a-d)} are significantly different.

Villi height to crypt depth (V/C) ratios were calculated by dividing the villi heights (μM) by the crypt depths (μM). Duodenum tissue from birds fed PB6 has the largest V/C ratio and was significantly larger than duodenum tissue obtained from both controls and N23 (1E6 CFU/g). Unchallenged birds had the smallest V/C ratio and were smaller than all but the challenged control. Jejunum tissue obtained from birds fed N23 (1E5 CFU/g) had the largest V/C ratios and were higher than both controls. Ilium tissues taken from birds fed N23 (1E5 CFU/g) had the greatest V/C ratios and were significantly more than the challenged control. Results are in Table 20.

TABLE 20
Villi height to crypt depth (V/C) ratios for duodenum, jejunum
and ilium sections of the small intestine obtained on day 42.

| | | Villi Height to Crypt Depth (V/C) Ratios | | |
|---|---|---|---|---|
| | | Duodenum | Jejunum | Ilium |
| 1 | Unchallenged | 12.84^{c} | 9.32^{c} | 9.58^{ab} |
| | Control | | | |
| 2 | Challenged | 17.54^{bc} | 12.55^{bc} | 8.74^{b} |
| | Control | | | |
| 3 | PB6 (1E5 CFU/g) | 27.61ª | 15.09^{ab} | 11.14^{ab} |
| 4 | N23 (1E5 CFU/g) | 22.89^{ab} | 18.43^{a} | 11.44^{a} |
| 5 | N23 (1E6 CFU/g) | 19.73^{b} | 17.21^{ab} | 11.37^{ab} |

Values not connected by the same letter^{(a-c)} are significantly different.

### Discussion

Bacillus velezensis N23 has shown superior in vitro efficacy⁵ against Salmonella, E. coli and select strains of Clostridia, making it a potential new direct-fed microbial that could add to Kemin's intestinal health portfolio. The biggest in vitro advantages with N23 over CLOSTAT® and ENTEROSURE® have been seen with Salmonella and E. coli⁵. Significant performance differences were not seen between N23 and PB6 but four-point improvement in adjusted FCR and a 130 g greater weight gain was seen with birds fed N23 when compared to birds fed PB6 fed at the same level (1E5 CFU/g) during the 42-day trial. Overall mortality was higher than what is typically seen in poultry (~5%) over 42 days. Brett Lumpkin, the study director at SPFR, stated that high heat and humidity contributed to mortality. The reason why the unchallenged control had the highest mortality is unknown but likely a product heat and humidity. Overall mortality, while high, did not significantly differ among groups. NE mortality rate was significantly lower with N23 and PB6 when compared to the challenged control and N23 NE mortality was numerically lower than PB6. Lower NE mortality rates in treated groups (1.9%; N23; 1E6 CFU/g, 2.4%; N23 1E5 CFU/g, and 2.6%; PB6 1E5 CFU/g) compared to the challenged control (5.4%) could mean greater profits for producers, so this finding is meaningful. Also, NE lesion scores were significantly reduced with all treatments for day 21 and with N23 (1E5 CFU/g) for day 35. Recovered N23 direct-fed microbial counts from feed were lower than target but performance results were in-line with PB6, which had counts that were in-line with expected counts. Lower N23 counts were likely due to lower treatment counts (Table 7). No differences were seen in microbiome alpha and beta diversity. During this study cecal samples were collected and the contents were analyzed for the fecal microbiome. This method proved beneficial with respect to DNA recovery from samples as all samples had sufficient DNA for sequencing. Previous NE and coccidiosis studies conducted in KANA R and D used excreta samples which often did not yield DNA even with multiple extraction attempts. The reason for this is unknown but many of the excreta samples that did yield DNA had the consistency of wet feed. By obtaining cecal content samples we corrected the extraction issues but we may not see the differences in microbiome that excreta samples taken earlier in the trial exhibit because the microbiomes of 42-day old birds are more mature.

Duodenum villi height from tissues obtained from birds fed PB6 were the highest indicating PB6 improves villi height and V/C ratios. Jejunum villi height was best in birds fed N23 and they also had the best V/C ratios. Ilium villi height was best in birds in the challenged control but they also had the largest crypts, so the V/C ratio was still smaller than all other groups. Samples (1 bird/pen; 70 pens) were obtained for tight junction protein testing.

### CONCLUSION

Significant performance differences were not seen between N23 and PB6 but a four-point improvement in adjusted FCR and a 130 g greater weight gain was seen with birds fed N23 when compared to birds fed PB6 fed at the same level (1E5 CFU/g) during the 42-day trial. A four-point improvement is not statistically significant, but to commercial poultry producer the financial impact may be. Reducing the amount of feed required to raise chickens for market reduces input costs and higher live weights means more money per bird, resulting in greater profit margins.

It should be appreciated that minor dosage and formulation modifications of the composition and the ranges expressed herein may be made and still come within the scope and spirit of the present invention.

Having described the invention with reference to particular compositions, theories of effectiveness, and the like, it will be apparent to those of skill in the art that it is not intended that the invention be limited by such illustrative embodiments or mechanisms, and that modifications can be made without departing from the scope or spirit of the invention, as defined by the appended claims. It is intended that all such obvious modifications and variations be included within the scope of the present invention as defined in the appended claims. The claims are meant to cover the claimed components and steps in any sequence which is effective to meet the objectives there intended, unless the context specifically indicates to the contrary.

The foregoing description has been presented for the purposes of illustration and description. It is not intended to be an exhaustive list or limit the invention to the precise forms disclosed. It is contemplated that other alternative processes and methods obvious to those skilled in the art are considered included in the invention. The description is merely examples of embodiments. It is understood that any other modifications, substitutions, and/or additions may be made, which are within the intended spirit and scope of the disclosure. From the foregoing, it can be seen that the exemplary aspects of the disclosure accomplish at least all of the intended objectives.

1. An isolated Bacillus velezensis strain having deposit accession number PTA-127793, or a mutant thereof having at least 95% sequence identity.

2. A direct-fed microbial composition comprising an isolated Bacillus velezensis strain having deposit accession number PTA-127793, or a mutant thereof.

3. The composition of claim 2, wherein the composition is an animal feed additive.

4. The animal feed according to claim 3, wherein the Bacillus velezensis PTA-127793 is present with a bacterial count between about 1×10³ and 1×10¹⁴ CFU/g of animal feed.

5. The animal feed or animal feed additive according to claim 3, further comprising a carrier.

6. The animal feed or animal feed additive according to claim 5, wherein the carrier comprises one or more of the following compounds: water, silica, glycerol, ethylene glycol, 1,2-propylene glycol or 1,3-propylene glycol, sodium chloride, sodium benzoate, potassium sorbate, sodium sulfate, potassium sulfate, magnesium sulfate, sodium thiosulfate, calcium carbonate, sodium citrate, dextrin, maltodextrin, white oil, corn cob meal, rice bran, glucose, sucrose, sorbitol, lactose, wheat flour, wheat bran, corn gluten meal, starch and cellulose.

7. The animal feed or animal feed additive of claim 3, further comprising at least one enzyme, prebiotic, probiotic, supplement, or milk replacer.

8. The composition of claim 2, wherein Bacillus velezensis PTA-127793 is present as spores.

9. A method of inhibiting or reducing pathogen growth in animal feed comprising adding a composition comprising Bacillus velezensis PTA-127793 to the animal feed.

10. The method of claim 9, further comprising the step of administering the composition to an animal, wherein the administration improves one or more performance parameter of the animal.

11. The method of claim 9, wherein the pathogen is selected from the group consisting of E. coli, Salmonella, Streptococcus, Rhodococcus, Enterococcus, and Clostridium.

12. The method of claim 10, wherein the one or more performance parameter is selected from the group consisting of body weight gain, European production efficiency factor, feed conversion ratio, animal health, and egg yield.

13. The method of claim 12, wherein improving the health of the animal comprises reducing mortality in the animal as compared to an animal not administered the strain.

14. The method of claim 9, wherein the administering prevents necrotic enteritis in the animal.

15. The method of claim 9, wherein the administering reduces or ameliorates symptoms associated with a pathogenic infection in the animal.

16. The method of claim 9, wherein the administering decreases antibiotic use in an animal production system.

17. The method of claim 9, wherein the Bacillus velezensis PTA-127793 is present as spores.

18. The method of claim 9, wherein the animal is selected from the group consisting of bovine, equine, poultry, fish, and shellfish.

19. The method of claim 9, wherein the composition further comprises at least one of an animal feed ingredient, a supplement, a milk replacer, an enzyme, a prebiotic, and/or a probiotic.

20. The method of claim 9, wherein the composition is fed to the animal, at a rate of about 1.0 g of Bacillus velezensis PTA-127793/head animal/day.
